(19) United States
(12) Patent Application Publication
(10) Pub. No.: US 2025/0139283 A1
(43) Pub. Date: May 1, 2025

(54) Distributed, secured, permissioned, multi-device connected system and method empowering the patients and guardians to create, store, manage, and fully own their healthcare records for generations

(71) Applicants: Sai Keertika Reddy Dorasani, Maryland Hts, MO (US); Tanish Asesa Reddy Dorasani, Maryland Hts, MO (US)

(72) Inventors: Sai Keertika Reddy Dorasani, Maryland Hts, MO (US); Tanish Asesa Reddy Dorasani, Maryland Hts, MO (US)

(21) Appl. No.: 18/622,978

(22) Filed: Mar. 31, 2024

Related U.S. Application Data
(60) Provisional application No. 63/493,751, filed on Apr. 1, 2023.

(51) Int. Cl. G06F 21/62; G06F 9/451; G06F 21/60; G06F 21/64; G16H 10/60

(52) U.S. Cl. CPC G06F 21/6245; G06F 9/451; G06F 21/602; G06F 21/64; G16H 10/60

(57) ABSTRACT

The present invention, the Vaidyo Bioblockchain System, addresses the significant challenges encountered in storing, sharing, and maintaining patient records across various platforms, including multiple patient portals and physical documents. Based on the difficulties parents faced managing healthcare information, the inventors developed this system to revolutionize the traditional, unstructured methods of healthcare data management. Central to this invention is the resolution of data ownership issues prevalent in existing healthcare record systems, where patient data is typically stored within organizational domains, thereby allowing these entities to benefit from the information. In contrast, the Vaidyo Bioblockchain System innovatively grants patients full ownership and control over their data, ensuring it is stored in a patient-authorized space. This system not only simplifies the process of managing healthcare information but also fundamentally shifts the dynamics of data ownership, empowering patients by placing them at the center of their healthcare data ecosystem.

A distributed, secured, permissioned, multi-device connected system and method empowering the patients and guardians to create, store, manage, and fully own their healthcare records for generations.

### FIELD OF THE INVENTION

This invention relates to digital healthcare management systems, specifically focusing on a distributed, permission-based software and multi-device platform designed to enhance the security, accessibility, and interoperability of healthcare data. It addresses the pervasive challenges in the healthcare industry regarding the efficient storage, management, and secure access to health records across a diverse ecosystem that includes computers, embedded devices, smart devices, and wearables. Unlike existing systems that often leave patients navigating fragmented and siloed health information, the “Vaidyo Bioblockchain” system introduces a revolutionary approach that empowers patients and guardians with ownership and comprehensive control over their health data. By leveraging advanced distributed ledger technology, the invention ensures that healthcare data is not only secure and immutable but also seamlessly accessible, fostering a more integrated, patient-centric healthcare experience. This innovation significantly contributes to the fields of health data management and medical informatics, offering a scalable solution to modernize health recordkeeping, enhance patient care, and support the broader goals of medical research and public health.

### DESCRIPTION OF THE RELATED ART

In the realm of healthcare management, the advent of Electronic Health Records (EHRs) marked a significant leap toward digitization. However, this transition has not been without challenges. Patients and healthcare providers alike grapple with a fragmented healthcare data landscape characterized by a disjointed assortment of physical documents, multiple patient portals, and a variety of electronic systems, each employing different formats and languages. This fragmentation severely hampers the continuity of care, complicating the comprehensive compilation of a patient's health history.

A critical obstacle within this landscape is the scattered nature of health records. Patients frequently encounter difficulties in consolidating their health information, often distributed across various healthcare providers, lost, or trapped in inaccessible formats. This issue is exacerbated by the healthcare sector's heightened vulnerability to data breaches, posing a significant threat to the privacy and security of sensitive health information. The recurring incidents of such breaches further complicate patients' efforts to manage their health records securely and cohesively.

Moreover, the existing systems largely place the ownership of health data within the domain of healthcare organizations rather than with the patients themselves. This arrangement restricts patients' access to and control over their own health information, thereby limiting their ability to manage their healthcare effectively. The absence of a unified system interface amplifies these challenges, making it almost impossible for patients and guardians to maintain a coherent electronic document chain of their and their families' health progression.

The “Vaidyo Bioblockchain” system emerges as a response to these multifaceted challenges, aiming to redefine the management, accessibility, and security of healthcare records. By placing patients at the center of the data ownership model and leveraging distributed ledger technology, the invention seeks to overcome the limitations of the current state of healthcare data management.

### Challenges Addressed By the Invention

The healthcare sector's transition to digital record-keeping, while beneficial, has introduced several challenges that undermine the efficiency, security, and accessibility of patient healthcare information. The “Vaidyo Bioblockchain” system is engineered to address these critical issues:

1. Fragmentation of Healthcare Records: Patients often struggle with health records scattered across various providers and platforms, making it difficult to compile a complete health history. This fragmentation hampers the continuity and quality of care, leading to challenges in diagnosing and treating conditions effectively.
2. Data Security and Privacy Concerns: The frequent occurrence of data breaches in the healthcare sector raises significant concerns about the security and privacy of patient information. Existing systems have proven inadequate in protecting sensitive health data against unauthorized access and cyber threats.
3. Limited Patient Control Over Health Data: Traditional healthcare record systems centralize data ownership with healthcare organizations, restricting patients' access to and control over their own health information. This limitation affects patients' ability to manage their healthcare and make informed decisions.
4. Inefficiency and Inaccessibility: The lack of a unified system interface complicates the process of accessing and sharing health records, especially in urgent care situations. Patients and healthcare providers face unnecessary delays and obstacles in accessing critical health information.

### Objectives of the Invention

In response to the challenges in healthcare, the “Vaidyo Bioblockchain” system aims to:

1. Centralize and Secure Health Records: Create a unified platform that consolidates health records from various sources into a single, secure repository. Leveraging blockchain technology, the system ensures the immutability and high-level security of stored data.
2. Empower Patients with Data Ownership: Shift the paradigm of health data ownership to patients, enabling them to exercise full control over their health records. This empowerment includes granting and revoking access to healthcare providers and family members as needed.
3. Enhance Accessibility and Interoperability: Ensure seamless access to health records across multiple devices and platforms, improving the interoperability between different healthcare systems. The system facilitates easy sharing and management of health records, improving the efficiency of healthcare delivery.
4. Contribute to Medical Research: Incorporate an incentivization mechanism that encourages patients to share anonymized health data with research institutions. This sharing aims to advance medical research while respecting patient privacy and ensuring data security.

Through these objectives, the “Vaidyo Bioblockchain” system introduces a transformative solution to the prevailing issues in healthcare data management. It not only addresses the immediate challenges of security, accessibility, and patient empowerment but also lays the groundwork for a more integrated, efficient, and patient-centered healthcare ecosystem.

### BRIEF SUMMARY OF THE INVENTION

The “Vaidyo Bioblockchain” system emerges as an innovative solution in the digital healthcare management landscape, aiming to fundamentally transform how healthcare records are stored, managed, accessed, and shared. It addresses the critical challenges that patients and guardians face in accessing health records, which are often scattered across various formats and locations, by introducing a unified, secure, and permission-based platform.

A primary objective of this invention is to facilitate a more efficient and user-friendly healthcare experience by enabling the creation, storage, and management of health records in a singular interface. This system is designed with advanced permission settings that can be finely tuned by patients, allowing them to grant access to other users, such as family members or healthcare providers, who play a role in the patient's care. This not only enhances the accessibility and continuity of care but also significantly improves the security and privacy of sensitive health data.

Moreover, “Vaidyo Bioblockchain” empowers patients and guardians with ownership over their health records, paving the way for successive generations to benefit from a comprehensive health history. This aspect of the invention is crucial for the early discovery of hereditary diseases, enabling timely diagnoses, more effective treatment plans, and potentially preventing diseases known to run in families. By providing a platform that ensures the integrity, accessibility, and continuity of health records, the invention stands to make a significant impact on individual and public health outcomes.

Additionally, the system introduces a novel approach to medical research participation. Patients and guardians can opt to share anonymized health data with research institutions, contributing to the advancement of medical knowledge and treatment methodologies. This not only aids in the progression of healthcare but also rewards participants, fostering a sense of contribution towards the greater good. In essence, the “Vaidyo Bioblockchain” is more than a digital healthcare management system; it is a comprehensive ecosystem that supports the healthcare journey of individuals and families, ensuring that health records are secure, accessible, and beneficial for current and future generations. It signifies a major leap forward in addressing the long-standing issues of health record management, privacy, and patient empowerment in the healthcare sector.

### BRIEF DESCRIPTION OF THE DRAWINGS

The drawings referred to here in the current application present the main objectives in the form of embodiments of the invention currently disclosed. The drawing embodiments are some of the key parts of the invention but invention features are not limited by only these drawings. All the embodiments are represented by figures.

FIG. 1: The patient struggles to locate the health records

### The Figure Includes:

Center Figure: A stick figure representing the patient, indicating the central role of the patient in the healthcare system.
Family Records (Top Left): An icon depicting a group of people and a document, representing issues patients face with managing and accessing family health records.
Paper Records (Bottom Left): An icon showing paper documents, illustrating the challenge of maintaining and organizing physical health records.
Multiple Portals (Top Center): An icon with interconnected computers, highlighting the complication of accessing health information through various online portals.
Data Breaches (Top Right): An icon with a lock and a warning sign, indicating concerns related to the security and privacy of health data.
Data Ownership (Bottom Right): An icon with a database and a person, symbolizing the issue of patient ownership and control over their health data.

FIG. 2: Vaidyo Bioblock Chain Illustrating Lifespan Health Record Continuity
9 Months Old: An icon representing a baby, indicating the start of the health record chain at an early age.
1 Year Old: An icon depicting a young child, signifying the continuation of health data recording from infancy.
5 Years Old: An icon showing a preschool-aged child, illustrating the growth and ongoing documentation of health records.
13 Years Old: An icon representing a teenager, marking the adolescence stage in the health record chain.
40 Years Old: An icon depicting an adult, indicating the mid-life stage and continued health data management.
70 Years Old: An icon representing an elderly person, signifying the comprehensive health record that spans the individual's entire life.
Connecting Blocks: Each age stage is connected by blocks representing the blockchain technology used to securely store and manage health records continuously throughout a person's life.

FIG. 3: Quick Summary of Vaidyo Bioblock Chain
Blocks: The figure shows three connected blocks, each representing a data block in the Vaidyo Bioblock Chain.
Hashes: Each block contains a hash value, which uniquely identifies the block's data.
   First Block: Hash: 12A8, Previous Hash: 0000 (indicating it is the first block in the chain).
   Second Block: Hash: 634S, Previous Hash: 12A8 (linking it to the first block).
   Third Block: Hash: 1283a, Previous Hash: 634S (linking it to the second block).
Connections: The blocks are connected by chains, representing the linkage and continuity of the blockchain.
Previous Hashes: Each block includes a reference to the previous block's hash, ensuring the integrity and immutability of the blockchain.

FIG. 4: Proving the Consensus of the Vaidyo Bioblock Chain
Nodes: The figure illustrates multiple nodes (represented by people icons) involved in the consensus process.
Blocks: Each node has a series of blocks connected, representing their individual copy of the blockchain.
Arrows: Arrows between the nodes indicate the communication and agreement process to achieve consensus.
Consensus Label: The word “CONSENSUS” with a check mark above it signifies the successful agreement among the nodes on the state of the blockchain.

FIG. 5: Tracking the Family Health History
Family Tree Structure: The figure illustrates a family tree structure with multiple nodes representing individuals within a family.
Individual Nodes: Each node in the family tree represents a person, indicating their place within the family hierarchy.
Medical Condition Tracking: Dotted lines and an icon representing a document indicate the tracking of specific medical conditions across different family members.
Connection Lines: Solid lines represent familial relationships, while dotted lines highlight the tracking of medical conditions.

FIG. 6: System Architecture
Patient: The figure shows a patient interacting with the App to input and fetch their health data.
Doctor: The doctor interacts with the Provider/Hospital system to create bioblocks and fetch patient data.
Researcher: Researchers interact with the Research Company to fetch data for research purposes and provide rewards for data access.
Lab Technician: Lab technicians interact with Laboratories to fetch and update patient records.
Vaidyo API: Central to the system, the Vaidyo API manages the data flow between all the entities (App, Provider/Hospital, Research Company, Laboratories).
Data Flow:
   Send Data (Dashed Lines): Indicates the flow of data from the entities to the Vaidyo API.
   Fetch Data (Solid Lines): Indicates the retrieval of data from the Vaidyo API by the entities.
   Create Bioblock: The process by which the Provider/Hospital creates a bioblock in the Vaidyo system.
   Rewards: The system of incentives provided to researchers for contributing data.

FIG. 7: Vaidyo System App Signup
Signup Form: The figure illustrates the signup interface of the Vaidyo System App, which includes fields for user input.
Fields:
   First Name: A text input field for the user's first name.
   Last Name: A text input field for the user's last name.
   Gender: A text input field for the user's gender.
   Date of Birth: A text input field for the user's date of birth.
   Username: A text input field for the user's desired username.
   Password: A text input field for the user's password.
Submit Button: A button labeled “SUBMIT” for submitting the entered information to create a new account.

FIG. 8: Vaidyo System App creates a unique Bio ID
My Bio Section: The upper section of the interface where user details are displayed.
   Name: Displays the user's name (e.g., Sai Dorasani).
   Date of Birth: Displays the user's date of birth.
   Gender: Displays the user's gender.
My Biocard Section: Displays the user's Biochain ID and Username.
   Biochain ID: Displays the unique Biochain ID assigned to the user.
   Username: Displays the user's chosen username.
   QR Code: A QR code is displayed for quick access or sharing of Biochain ID.
My Biohealth Section: Displays the user's health metrics.
   Height: Displays the user's height.
   Weight: Displays the user's weight.
   BP: Displays the user's blood pressure.
   Oxygen: Displays the user's oxygen level.
Navigation Bar: Located at the bottom, providing navigation options.
   Biocard: Navigation option to view the Biocard

FIG. 9: Vaidyo System App for Booking Appointment
Book Appointment Interface: Displays the form used by users to book medical appointments within the Vaidyo System App.
   Your Doctor: A text input field where users can enter or select the name of the doctor they wish to book an appointment with.
   Available Time: A text input field where users can specify the times they are available for the appointment.
   Reason for Visiting and Current Symptoms: A text input field where users can describe the reason for their visit and list any current symptoms they are experiencing.
Submit Button: A button labeled “SUBMIT” that users click to finalize and submit their appointment request.
Navigation Bar: Located at the bottom of the interface, providing quick access to different sections of the app.
   Biocard: A navigation option to view the user's Biocard.
   Biochain: A navigation option to view the user's Biochain.
   Add Book: A navigation option to add new records or book an appointment.
   Rewards: A navigation option to view rewards available to the user.

FIG. 10: Vaidyo System Bioblocks for a Booked Appointment
My Biochain Section: Displays blocks of data related to the user's appointments and insurance.
   Data Block 1: Contains booking information.
      User ID: BCU-1
      Key: bookingInfo
      Value: Doctor: Tanish Asesa, Appointment Time: Nov. 1, 2022, 4 pm CST, Reason for Visit: Flu
      Hash: a61458a888c9ae9648e60be6621aa748
      Timestamp: 1667330406535
   Data Block 2: Contains insurance information.
      User ID: BCU-1
      Key: insurance
      Value: Blue Cross 17383
      Hash: 97155adc71d25b53d2ddd97ac07babb65
      Timestamp: 1667330400937
Navigation Bar: Located at the bottom, providing navigation options.
   Biocard: Option to view the Biocard.
   Biochain: Option to view the Biochain.
   Add Book: Option to add new records or book appointments.
   Rewards: Option to view rewards available to the user.

FIG. 11: Vaidyo System App Health Data Retrieval Interface
My Bio Section: Displays user details.
   Name: Sai Dorasani
   Date of Birth: User's date of birth.
   Gender: User's gender.
My Biocard Section: Displays the user's Biochain ID and Username.
   Biochain ID: Unique Biochain ID assigned to the user.
   Username: User's chosen username.
   QR Code: A QR code for quick access or sharing of the Biochain ID.
My Biohealth Section: Displays the user's health metrics as entered by the doctor.
   Height: 5.5
   Weight: 86
   BP: 130 (Blood Pressure)
   Oxygen: 98 (Oxygen level)
Navigation Bar: Located at the bottom, providing navigation options.
   Biocard: Navigation option to view the Biocard.
   Biochain: Navigation option to view the Biochain.
   Add Book: Navigation option to add new records or book appointments.
   Rewards: Navigation option to view rewards available to the user.

### DETAILED DESCRIPTION OF THE INVENTION

### Overview of the Invention

The “Vaidyo Bioblockchain” system is a pioneering digital healthcare management platform designed to revolutionize the way health records are stored, accessed, managed, and shared. By leveraging distributed ledger technology, the system ensures a high level of security, privacy, and interoperability across various digital devices and formats. This detailed description outlines the system's architecture, functionality, and user interface, emphasizing its innovative features and the benefits it offers to patients, guardians, and healthcare providers.

### System Architecture

The core of the “Vaidyo Bioblockchain” system is built on a distributed ledger framework, enabling secure and immutable storage of health records. The architecture supports a multi-device environment, including computers, smartphones, wearable devices, and embedded systems, ensuring seamless integration and accessibility.

Distributed Ledger Technology (DLT): Utilizes blockchain to create a secure, immutable history of health records.
Permissioned Access Control: Implements granular permissions for data access, controlled by the patient, allowing specific users to view or edit health records based on predefined criteria.
Multi-Device Compatibility: Ensures that the system is accessible across a wide range of devices, facilitating easy access and management of health records.

### Rewarding System Components

A crucial component of the “Vaidyo Bioblockchain” system is its innovative rewarding system, designed to actively engage patients in the healthcare data sharing process by providing incentives for their contributions to medical research. This aspect not only underscores the system's commitment to secure and patient-centric healthcare data management but also fosters a participatory healthcare ecosystem.

Reward Management Module: This dedicated module is pivotal for administering rewards to patients who voluntarily share their healthcare data. It meticulously tracks sharing activities and calculates rewards based on predefined parameters, such as the volume of data shared, frequency of sharing, and types of shared data.
Integration with Digital Wallets: To facilitate the seamless distribution and redemption of rewards, the Reward Management Module is integrated with digital wallets or points systems. This integration allows patients to receive rewards in various forms, including but not limited to digital tokens, points, or vouchers, which can be utilized within the healthcare ecosystem or exchanged for other benefits.
   Data Anonymization and Consent Layer: Central to the system's data sharing capability is the anonymization process, which strips personal identifiers from patient data before it is shared with third-party organizations for analytics. This layer is instrumental in preserving patient privacy. Furthermore, patients retain the ability to manage their consent through an intuitive interface, delineating precisely which data can be shared, with whom, and under what stipulations.
Dynamic Consent Interface: Enabling patients to adjust their data sharing preferences in real time effortlessly, this interface provides them with unfettered control over their healthcare data. Modifications to consent preferences and data access requests are immutably recorded on the blockchain, ensuring a transparent and auditable trail of permissions and data sharing activities.

By incorporating the rewarding system into the “Vaidyo Bioblockchain” system architecture, the platform not only secures and manages healthcare data but also actively engages patients in the data sharing process, rewarding them for their contributions to medical research. This approach fosters a participatory healthcare ecosystem where patient contributions are recognized and incentivized, enhancing the value of shared healthcare data for the greater good.

### Third-Party Integration for Data Sharing

In the evolving landscape of healthcare, the ability to securely and efficiently share patient data with third-party entities—such as research institutions, specialist healthcare providers, and analytics companies—has become paramount. The “Vaidyo Bioblockchain” system addresses this need through a sophisticated integration framework that ensures patient data is not only protected but also utilized to foster advancements in medical research and healthcare delivery.

Secure API Framework: At the heart of the system's third-party integration capabilities is a robust and secure application programming interface (API) framework. This framework enables seamless data exchange between the “Vaidyo Bioblockchain” and external systems, ensuring that all shared data is encrypted and transmitted over secure channels. The API is designed with flexibility in mind, allowing for compatibility with a wide range of healthcare applications and systems, thereby enhancing the ecosystem's overall interoperability.
Granular Consent Management: Central to the system's data sharing functionality is an advanced consent management module. This module empowers patients with the ability to precisely control how their health data is shared with third-party entities. Patients can grant or revoke consent for specific data sets, determine the scope of data access, and set time-bound permissions for data usage. This granular approach to consent ensures that patients retain sovereignty over their health information, aligning with the system's core principle of patient-centric data ownership.
Data Anonymization Protocols: Prior to sharing any patient data with third-party entities, the system employs stringent data anonymization protocols to strip away personal identifiers, thereby safeguarding patient privacy. This process ensures that the data shared for research or analysis purposes cannot be traced back to individual patients, thus maintaining confidentiality while contributing valuable insights to the medical community.
Collaborative Research and Analytics: By facilitating the secure and consented sharing of anonymized health data, the “Vaidyo Bioblockchain” system opens new avenues for collaboration between patients and the research community. Research institutions gain access to a rich repository of health data, enabling them to conduct studies that can lead to breakthroughs in understanding disease patterns, treatment effectiveness, and health trends. This collaborative model not only accelerates medical research but also positions patients as active contributors to the collective knowledge base of healthcare.

Through its comprehensive third-party integration for data sharing, the “Vaidyo Bioblockchain” system exemplifies a future where digital healthcare management transcends traditional boundaries, fostering an ecosystem of collaboration, innovation, and enhanced patient care.

### Functional Capabilities

Health Record Creation and Management: Users can easily create, upload, and manage their health records in various formats (e.g., text, images, videos). The system supports automatic translation and format conversion to standardize the records across different healthcare providers.
Granular Permission Settings: Allows patients to set detailed permissions for their health records, determining who can access or edit their information. This feature is critical for maintaining privacy and security.
Interoperability with Healthcare Providers: Integrates with existing healthcare systems and providers, enabling the seamless transfer of health records and ensuring that patients' health information is up-to-date and comprehensive.
Generational Health Record Access: Facilitates the creation of a family health history chain, allowing future generations to access their ancestors' health records, invaluable for the early detection and prevention of hereditary diseases.

### User Interface and Experience

Intuitive Dashboard: A user-friendly dashboard provides patients and authorized users with a comprehensive overview of their health records, including medical history, recent visits, lab results, billing details, and upcoming appointments.
Health Data Analytics: Incorporates advanced analytics tools that enable users to gain insights into their health trends, potential risk factors, and preventive measures.
Secure Communication Channels: Offers encrypted messaging and data sharing features, allowing patients to communicate securely with healthcare providers and family members.

### Incentivization and Contribution to Medical Research

Reward System: Introduces a reward mechanism for users who agree to share anonymized health data with research institutions, contributing to medical advancements while ensuring their privacy and data security.
Data Analytics for Research: Provides researchers with access to a vast repository of anonymized health data, facilitating the study of disease patterns, treatment outcomes, and healthcare trends.

### Implementation and Deployment

The deployment of the “Vaidyo Bioblockchain” system involves collaboration with healthcare providers, IT infrastructure providers, and regulatory bodies to ensure compliance with health data protection regulations and seamless integration into existing healthcare ecosystems.

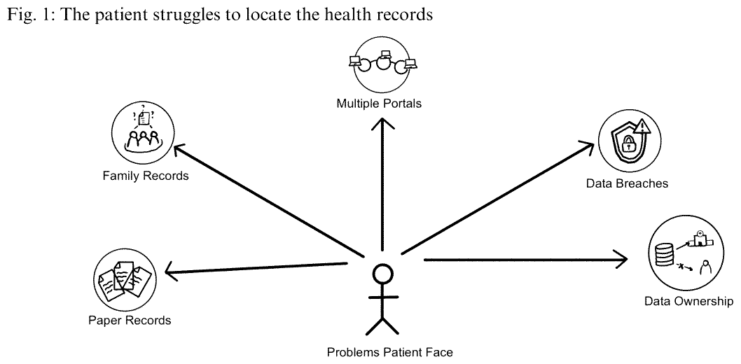

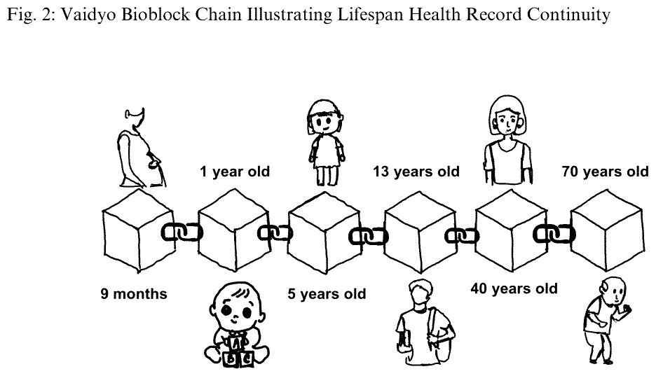

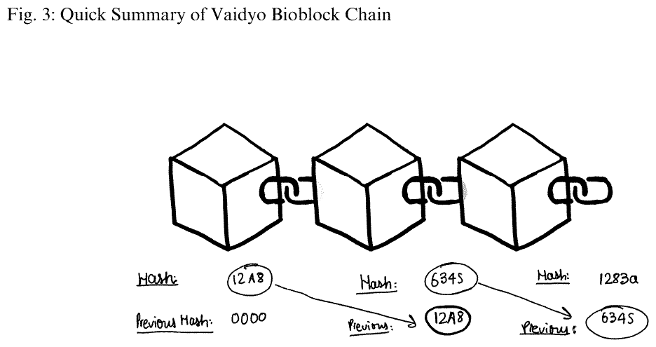

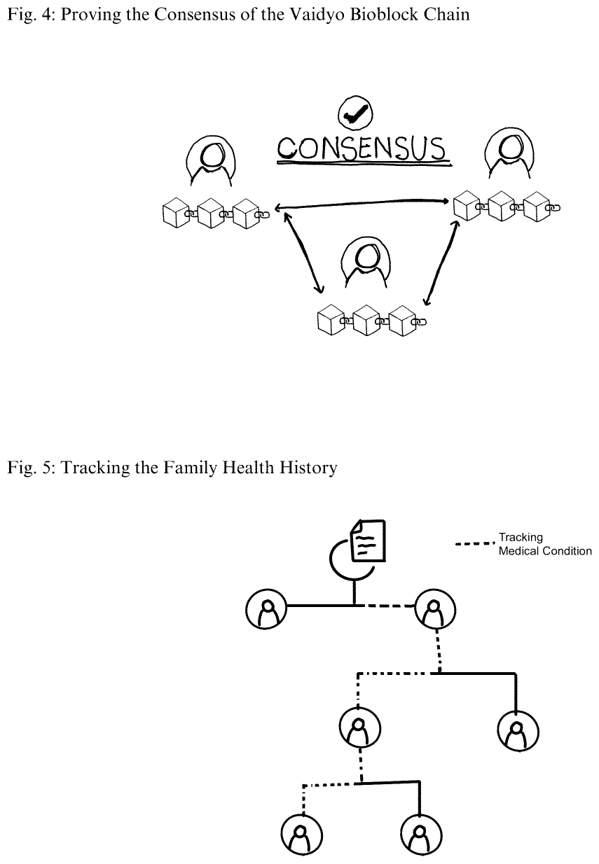

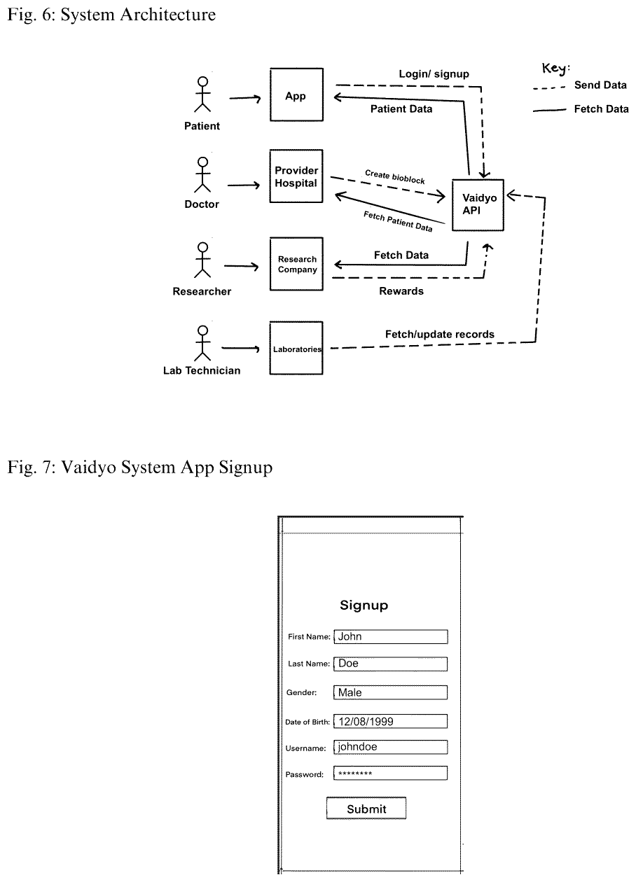

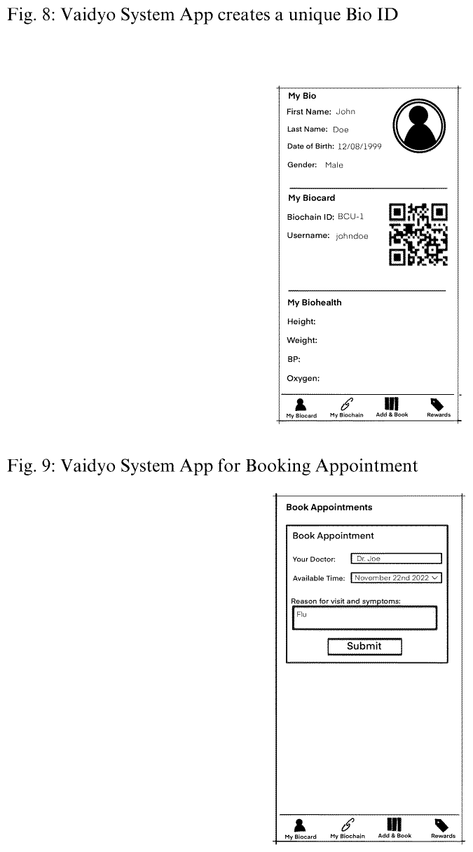

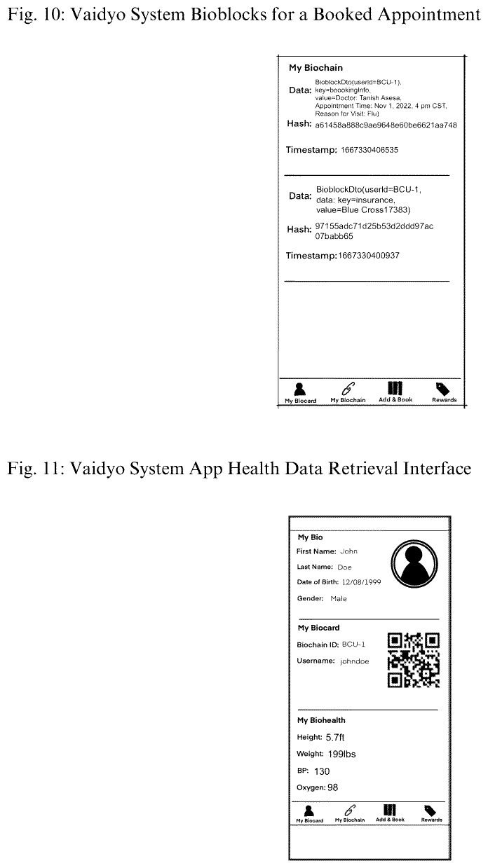

1: Enhanced Comprehensive Digital Healthcare Data Management System with Patient-Centric Data Ownership
A digital healthcare data management system, characterized by its patient-centric approach to data ownership, configured to:
Securely store healthcare records utilizing distributed ledger technology (DLT), ensuring data integrity and patient privacy through advanced cryptographic hashing and consensus mechanisms, establishing the foundation for patient ownership of healthcare data [refer to FIG. 3 & FIG. 4];
Provide a versatile user interface accessible across a variety of devices, enabling real-time access, creation, management, and sharing of healthcare records by patients, further reinforcing the patients' rights as primary owners and controllers of their health data [refer to FIG. 7 and FIG. 6];
Implement a permissioned access control module that empowers patients with the unequivocal authority to configure detailed access rights to their healthcare records, thereby granting or restricting access to different stakeholders, including healthcare providers and family members, based on patient-directed permissions, with functionalities for setting time-bound access, emphasizing the shift in data ownership from organizations to patients themselves;
Facilitate the integration and standardization of health records from disparate sources into a cohesive, unified digital health record system through the use of machine learning algorithms for effective translation and format standardization, enhancing data interoperability, accuracy, and ultimately ensuring that the control and ownership of healthcare data are vested in patients, thus fundamentally transforming the traditional dynamics of healthcare data management by centering it around patient autonomy [refer to FIG. 1].

2: Advanced Method for Multi-Device Healthcare Data Integration, Access, and Generational Health Management
A method for processing and accessing integrated healthcare data within a digital environment, characterized by its comprehensive and patient-centric approach, the method comprising steps to:
Aggregate and standardize healthcare records from a myriad of sources into a unified, digital health record system, employing Distributed Ledger Technology (DLT) to ensure secure, immutable record-keeping and facilitate the patient-centered ownership and control of health data;
Employ an advanced, adaptive user interface design optimized for seamless operation across a wide array of devices, including but not limited to computers, smartphones, wearable devices, and embedded systems, thereby enhancing real-time access, management, and sharing of health metrics and records; this interface integrates with wearable devices and other health monitoring equipment for continuous, real-time health status monitoring, promoting proactive patient health management and engagement;
Implement granular, customizable permission settings within the digital health record system, empowering patients with the ability to precisely specify and dynamically adjust access conditions for their healthcare data across different stakeholders-ranging from healthcare providers to family members-thus ensuring robust privacy controls and secure data sharing aligned with patient preferences and consent;
Facilitate the creation and maintenance of generational health record chains within the unified digital system, providing patients and their families access to comprehensive health histories that span multiple generations; this feature is instrumental in enabling the early detection of hereditary diseases, facilitating timely and accurate diagnoses, and informing preventive measures to mitigate potential future health issues; the system's emphasis on generational health data not only aids in the preservation of familial health legacies but also contributes significantly to personalized medicine and family health management strategies [refer to FIG. 2 & FIG. 5].

3: Advanced System for Incentivized Health Data Sharing and Research Contribution with Enhanced User Engagement and Data Security,
This system, designed to foster the anonymized sharing of health data for research purposes, is characterized by a comprehensive approach that includes privacy protection, collaborative research engagement, and an incentivization mechanism; the system comprises:
An integrated incentive mechanism that rewards users with digital tokens or credits for sharing their anonymized health data with research entities, where the rewards system is dynamically calibrated based on the quality, volume, and relevance of the shared data to ongoing research studies, encouraging sustained participation and contribution;
Sophisticated data anonymization protocols that utilize state-of-the-art encryption and data processing techniques to ensure the complete removal of personally identifiable information from health data prior to sharing, thereby upholding stringent data privacy standards and fostering user trust;
Secure Application Programming Interfaces (API) integrations facilitating the encrypted transmission of anonymized health data to a network of authorized research institutions and partners, ensuring data security during transmission and access, while supporting a seamless exchange of valuable health information for research purposes;
A collaborative framework for medical research that not only enables users to contribute to health studies but also integrates a transparent feedback loop, providing users with insights into the research outcomes and findings derived from their shared data, enhancing the visibility of the tangible impacts of their contributions on medical advancements;
The system further includes a user-centric portal that allows participants to manage their data sharing preferences, view ongoing research projects eligible for data contribution, and track their rewards accumulation, thereby promoting an active and informed user engagement in the research contribution process;
Additionally, the system implements a modular research participation interface, enabling researchers to specify data requirements for studies, facilitating targeted data collection efforts that align with the specific needs of medical research, thereby optimizing the utility and relevance of shared health data for advancing healthcare knowledge and innovation.
Dependent claims

4: The system of claim 1, wherein the secure, patient-owned healthcare data repository utilizes cryptographic hashing and consensus mechanisms for robust data validation and protection, reinforcing the patient's role as the sovereign owner of their health records; the system further supports the creation of a generational health record chain, ensuring that patients can establish and maintain a continuous and secure family health history, enabling long-term health monitoring and the early detection of genetic conditions; additionally, the system's user interfaces are meticulously optimized for both mobile and desktop platforms, featuring adaptive layouts and functionalities tailored to each device type, empowering patients with seamless and flexible access to their health data across devices, while maintaining their position as the ultimate custodians of their health information [refer to FIG. 3, FIG. 4, FIG. 8, and FIG. 9].

5: The system of claim 1, wherein the patient-centric data ownership model is augmented by an integrated appointment scheduling module, designed to empower patients with autonomous management of their healthcare engagements, enabling them to directly book, modify, and cancel appointments through a user interface that is synchronized in real-time with both patient and provider calendars; this system feature is complemented by a permissioned access control module, which entrusts patients with the exclusive ability to grant time-bound access to their health records for healthcare providers, thereby facilitating a secure exchange of health information tailored to each medical consultation; such meticulous control reinforces the patient's privacy and bolsters the security of their personal health information, in alignment with the fundamental principle that patients are the unequivocal owners of their healthcare data and have the right to determine how and when it is accessed [refer to FIG. 6 and FIG. 7].”

6: The system of claim 1, further refined to empower patients with exclusive data ownership, including a feedback mechanism within the user interface that allows patients to directly report inaccuracies, request updates, and assertively manage their healthcare records, thus ensuring the integrity and accuracy of their health information; the system additionally features personalized health analytics tools that generate insights and recommendations, supporting patients in preventive care decision-making processes; these features collectively affirm the patients' status as the principal owners and beneficiaries of their health data, shifting the paradigm from organizational data control to patient-centric data stewardship and autonomy [refer to FIG. 10 and FIG. 11].

7: The method of claim 2, involving the use of machine learning algorithms for the identification, merging, and resolving of duplicates in healthcare records from multiple sources, including the translation and standardization of these records into preferred languages and formats for global interoperability and data sharing, further extending to real-time health metrics monitoring through wearable device integration and dynamic consent management for real-time access permission adjustments [refer to FIG. 2 and FIG. 5].

8: The method of claim 2, further including automatic updates of patient health records with new data entries from healthcare providers, labs, and imaging centers, coupled with a predictive health analytics feature that leverages real-time and historical health data for potential health risk identification and notification to patients and healthcare providers.

9: The method of claim 2, additionally includes leveraging advanced data analytics to provide users with actionable health insights based on their aggregated and standardized health records, facilitating personalized health management strategies and enabling proactive patient engagement in their healthcare journey.

10: The method of claim 2, extends to integrate a seamless communication channel within the user interface, enabling secure and encrypted interactions between patients and healthcare providers, thus enhancing the collaborative care model by streamlining consultations, feedback, and follow-up actions based on real-time health data analysis.

11: The method of claim 2, further includes the process of automatically updating patient health records with new data entries from healthcare providers, labs, and imaging centers, ensuring a continuously updated and comprehensive health record.

12: The method of claim 2, extends to incorporate a predictive health analytics feature that analyzes real-time health metrics in conjunction with historical health data to identify potential health risks and notify patients and healthcare providers.

13: The method of claim 2, wherein integrating healthcare data from multiple sources involves utilizing machine learning algorithms to identify, merge, and resolve duplicates across disparate data sets, ensuring data accuracy and completeness.

14: The method of claim 2, wherein the standardization of healthcare records includes aligning the data with international health data standards and formats, facilitating global interoperability and data sharing.

15: The system of claim 3, where the incentive mechanism includes a rewards system with digital tokens redeemable within a healthcare ecosystem, supported by secure API integrations for encrypted data transmission and data anonymization protocols, ensuring the ethical use of shared anonymized data for medical research, complemented by a participant dashboard for tracking and feedback, fostering transparency and engagement [refer to FIG. 6].

16: The system of claim 3, further comprises a comprehensive data privacy and security framework that employs state-of-the-art encryption methods for all data transmissions between the system and third-party research entities, ensuring that participant data sharing is conducted within a secure digital environment, safeguarding against unauthorized access and data breaches.

17: The system of claim 3, also includes a modular research participation interface that allows users to selectively engage with various research studies, providing them with detailed information about the study's goals, the specific data required, and the impact of their contribution, thereby empowering users with the choice and knowledge to make informed decisions about their participation in advancing medical research.

18: The system of claim 3, ensures that all shared anonymized data is subject to rigorous ethical review and is used solely for the purpose of advancing public health knowledge, with mechanisms in place to audit and verify the use of data by research institutions.

19: The system of claim 3, further comprises a feedback loop where participants can receive summaries of research findings and insights derived from their shared data, fostering transparency and participant engagement in medical research.

20: The system of claim 1, wherein an integrated solution module is configured to:
Centralize healthcare records from multiple healthcare portals and physical paper records into a single, patient-controlled digital repository, thereby simplifying patient access and management of their health records;
Enhance data security measures, thereby protecting against breaches and unauthorized access to safeguard patient data;
Establish a patient-centric data ownership framework that acknowledges patients as the unequivocal owners of their healthcare data, empowering them to control access to their records exclusively;
Provide the capability to store, organize, and maintain family health history, thus enabling the early detection of hereditary conditions and facilitating the proactive management of potential future health risks;
Integrate said solution module into the system's user interface to ensure it is readily accessible and user-friendly across the various devices supported by the system, directly addressing the problems depicted in FIG. 1.
